(19) United States
(12) Patent Application Publication
(10) Pub. No.: US 2020/0226755 A1
(43) Pub. Date: Jul. 16, 2020

(54) IMAGE PROCESSING APPARATUS, OPHTHALMIC IMAGING APPARATUS, IMAGE PROCESSING METHOD, AND COMPUTER-READABLE MEDIUM

(71) Applicant: CANON KABUSHIKI KAISHA, Tokyo (JP)

(72) Inventors: Yuki Shimozato, Hachioji-shi (JP); Wataru Sakagawa, Kawasaki-shi (JP)

(21) Appl. No.: 16/829,408

(22) Filed: Mar. 25, 2020

Related U.S. Application Data
(63) Continuation of application No. PCT/JP2018/035432, filed on Sep. 25, 2018.

(30) Foreign Application Priority Data
Sep. 29, 2017 (JP) 2017-190212

(51) Int. Cl. G06T 7/00; G06T 5/50; G06T 7/11; A61B 3/10; A61B 3/00; A61B 3/12

(52) U.S. Cl. CPC G06T 7/0012; G06T 5/50; G06T 7/11; G06T 2207/30041; A61B 3/0091; A61B 3/12; G06T 2207/10101; A61B 3/102

(57) ABSTRACT

Provided is an image processing apparatus including: an acquiring unit configured to acquire a plurality of pieces of tomographic data indicating tomographic information at substantially the same position of a subject to be inspected; and an image generating unit configured to generate a motion contrast image using the plurality of pieces of tomographic data, in which a data amount of a plurality of tomographic data used in generating one motion contrast image when the motion contrast image is to be generated as a moving image is smaller than a data amount of a plurality of tomographic data used in generating one motion contrast image when the motion contrast image is to be generated as a still image.

### CROSS-REFERENCE TO RELATED APPLICATIONS

This application is a Continuation of International Patent Application No. PCT/JP2018/035432, filed Sep. 25, 2018, which claims the benefit of Japanese Patent Application No. 2017-190212, filed Sep. 29, 2017, both of which are hereby incorporated by reference herein in their entirety.

### BACKGROUND OF THE INVENTION

### Field of the Invention

The present invention relates to an image processing apparatus, an ophthalmic imaging apparatus, an image processing method, and a computer-readable medium.

### Description of the Related Art

As ophthalmic apparatus configured to capture tomographic images of an eye to be inspected, there are known apparatus (OCT apparatus) using optical coherence tomography (OCT). Further, in recent years, an image relating to a blood current in a fundus can be generated with the use of the tomographic images to acquire an image similar to an image in a related-art fundus fluorescein angiography. This technology is generally called OCT angiography (OCTA). An image acquired with the use of OCTA is hereinafter referred to as an “OCTA image”.

In OCTA, an interference signal at the same part of the eye to be inspected is acquired a plurality of times to generate a plurality of tomographic images. Thereafter, a change in luminance value at the same part (same pixel) in a cross section between the tomographic images is obtained as an image. It is known that the luminance of the interior of a blood vessel changes between tomographic images captured at different times because positions of blood cells in the blood vessel change.

In calculating the change in luminance value, there are used various calculation methods including determining a variance of luminance values of pixels in the tomographic images that correspond to a pixel for which a pixel value is to be determined, or determining a decorrelation value between two tomographic images. In this specification, an image obtained based on an amount of change in luminance value of the tomographic images is called an “OCTA tomographic image”, and the amount of change in luminance value is called a “motion contrast value”. Further, an image generated with the use of the motion contrast value (motion contrast data) is collectively called a “motion contrast image”.

After an OCTA tomographic image is generated with the use of tomographic images at one position, OCTA tomographic images can be generated similarly for tomographic images obtained by sequentially changing the position in a normal direction of the cross section to produce three-dimensional OCTA volume data. An image obtained by projecting the three-dimensional OCTA volume data in in-plane directions (axial direction of main scanning and axial direction of sub-scanning) of the cross section is called an “OCTA image” or “OCTA front image”.

In Japanese Patent Application Laid-Open No. 2016-10656, there is proposed an apparatus in which, during OCTA imaging of a position, signal processing for generating an OCTA image at another position that has already been acquired is started to speed up display of OCTA images.

In general, it is known that signal processing for generating OCTA images takes longer time than signal processing for generating OCT tomographic images. Meanwhile, considering that the OCTA images are images relating to blood currents, it is considered that displaying a moving image of the OCTA images is also advantageous for diagnosis and other purposes. However, when the moving image of the OCTA images is to be generated, it is required to generate continuous images, and it takes more time than generating a still image.

To address the above-mentioned problem, it is an object of the present invention to provide an image processing apparatus, an ophthalmic imaging apparatus, an image processing method, and a computer-readable medium with which generation of a moving image of OCTA images can be sped up.

### SUMMARY OF THE INVENTION

According to one aspect of the present invention, there is provided an image processing apparatus including: an acquiring unit configured to acquire a plurality of pieces of tomographic data indicating tomographic information at substantially the same position of a subject to be inspected; and an image generating unit configured to generate a motion contrast image using plurality of pieces of tomographic data, wherein a data amount of a plurality of pieces of tomographic data used in generating one motion contrast image when the motion contrast image is to be generated as a moving image is smaller than a data amount of a plurality of pieces of tomographic data used in generating one motion contrast image when the motion contrast image is to be generated as a still image.

According to another aspect of the present invention, there is provided an ophthalmic imaging apparatus including: an imaging optical system configured to image substantially the same position of a subject to be inspected a plurality of times using measurement light to acquire a plurality of pieces of tomographic information at substantially the same position; an acquiring unit configured to acquire a plurality of pieces of tomographic data indicating the plurality of pieces of tomographic information; and an image generating unit configured to generate a motion contrast image using the plurality of pieces of tomographic data, wherein a data amount of a plurality of pieces of tomographic data used in generating one motion contrast image when the motion contrast image is to be generated as a moving image is smaller than a data amount of a plurality of pieces of tomographic data used in generating one motion contrast image when the motion contrast image is to be generated as a still image.

According to still another aspect of the present invention, there is provided an image processing method including: acquiring a plurality of pieces of tomographic data indicating tomographic information at substantially the same position of a subject to be inspected; and generating a motion contrast image using the plurality of pieces of tomographic data, wherein a data amount of a plurality of pieces of tomographic data used in generating one motion contrast image when the motion contrast image is to be generated as a moving image is smaller than a data amount of a plurality of pieces of tomographic data used in generating one motion contrast image when the motion contrast image is to be generated as a still image.

Further features of the present invention will become apparent from the following description of exemplary embodiments with reference to the attached drawings.

### BRIEF DESCRIPTION OF THE DRAWINGS

FIG. 1 is a diagram for illustrating an example of a schematic configuration of an OCT apparatus.

FIG. 2 is a diagram for illustrating an example of a schematic configuration of a control unit.

FIG. 3 is a diagram for illustrating an example of screen display.

FIG. 4A is a diagram for illustrating an example of OCT signal acquisition conditions.

FIG. 4B is a graph for showing an example of OCT signal acquisition conditions.

FIG. 5 is a flow chart for illustrating an example of an OCT signal acquiring sequence.

FIG. 6 is a flow chart for illustrating an example of an OCTA signal processing sequence.

FIG. 7 is a flow chart for illustrating an example of an imaging sequence in Embodiment 1 of the present invention.

FIG. 8 is a flow chart for illustrating an example of an imaging sequence in Embodiment 2 of the present invention.

FIG. 9 is a flow chart for illustrating an example of an imaging sequence in Embodiment 3 of the present invention.

### DESCRIPTION OF THE EMBODIMENTS

Preferred embodiments of the present invention will now be described in detail in accordance with the accompanying drawings.

Matters described in the following embodiments, which include dimensions, materials, shapes, and relative positions of components, are freely set, and can be changed depending on various conditions or configurations of apparatus to which the present invention is applied. In the drawings, the same reference symbols are used to denote components that are the same as one another or functionally similar to one another among the drawings. In this specification, the term “real-time display” refers to displaying, substantially at the same time as capturing, an image generated with the use of a signal obtained by the capturing.

### Embodiment 1

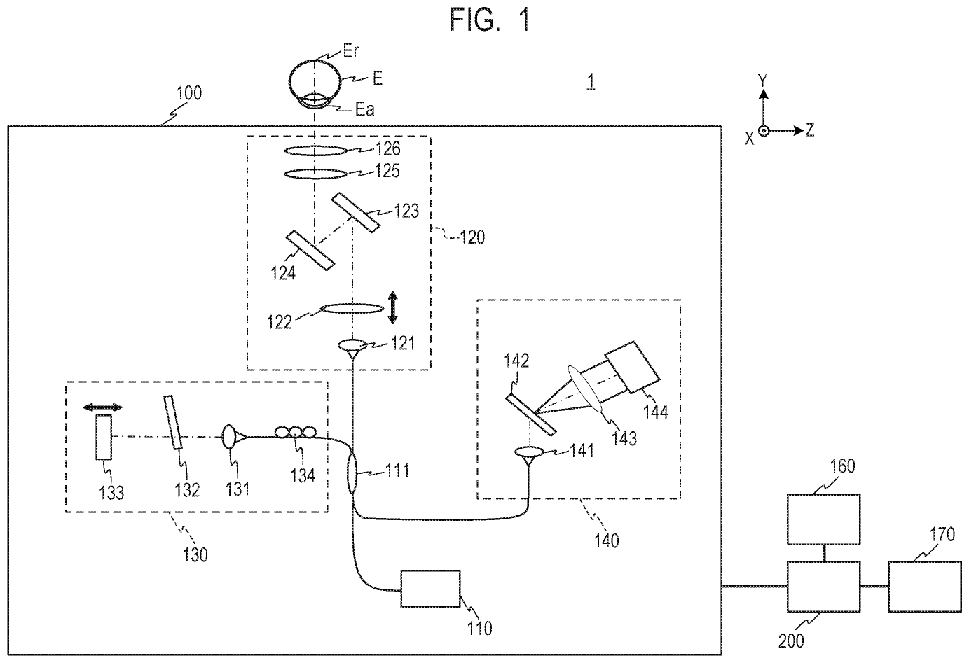

Now, referring to FIG. 1 to FIG. 7, an OCT apparatus 1 as an example of an ophthalmic imaging apparatus according to Embodiment 1 of the present invention, and steps of an image processing method executed in the OCT apparatus 1 are described. In the OCT apparatus 1 in Embodiment 1, an amount of acquired signals relating to OCTA imaging is changed for generation of a moving image and generation of a still image of OCTA images to speed up the generation of the moving image of the OCTA images. First, referring to FIG. 1 to FIG. 3, a schematic configuration of the OCT apparatus is described. FIG. 1 is a diagram for illustrating a schematic configuration of the OCT apparatus 1 in Embodiment 1.

<Configuration of OCT Apparatus>

In OCT, based on interference light obtained by causing return light from an eye to be inspected E irradiated with measurement light through a scanning unit, and reference light corresponding to the measurement light to interfere with each other, tomographic images of the eye to be inspected can be acquired. The OCT apparatus 1 in Embodiment 1 includes an imaging apparatus unit 100 (imaging optical system), a control unit 200 (image processing apparatus), a display unit 160, and an operation input unit 170.

The imaging apparatus unit 100 includes a measurement optical system arranged to capture a two-dimensional image and a tomographic image of an anterior eye Ea or a fundus Er of the eye to be inspected E. In generating the OCTA images, the imaging apparatus unit 100 is used to image substantially the same position (substantially the same part) of a subject to be inspected a plurality of times with the use of the measurement light based on light from a light source, and acquire a plurality of pieces of tomographic information of substantially the same position. The control unit 200 is connected to the imaging apparatus unit 100, the display unit 160, and the operation input unit 170. The control unit 200 can generate, based on various signals output from the imaging apparatus unit 100, two-dimensional images, tomographic images, OCTA images, and other images of the anterior eye Ea or the fundus Er of the eye to be inspected E. The control unit 200 may be formed with the use of a general-purpose computer, or as a computer specialized for the OCT apparatus 1.

The display unit 160 can display patient information, various images, and other information output from the control unit 200. The operation input unit 170 can be formed with the use of any input device, for example, a keyboard and a mouse, and an examiner can use the operation input unit 170 to input, to the control unit 200, the patient information, an imaging mode, an imaging range, various conditions on imaging, and other information. In this specification, the imaging apparatus unit 100, the control unit 200, the display unit 160, and the operation input unit 170 are separately provided, but a part or all of those units may be integrally formed.

Now, a configuration of the imaging apparatus unit 100, a configuration of the control unit 200, and display contents of the display unit 160 are described in the stated order.

<Configuration of Imaging Apparatus Unit 100>

The imaging apparatus unit 100 includes a light source 110, a coupler 111, a sample optical system 120, a reference optical system 130, and an interference optical system 140.

<Light Source 110>

The light source 110 is a super luminescent diode (SLD), which is a low coherent light source, and has a center wavelength of 855 nm and a wavelength bandwidth of about 100 nm. The bandwidth affects a resolution in an optical axis direction of the obtained tomographic images. Further, as the type of the light source, the SLD is selected in this example, but any other light source capable of emitting low coherent light may be used. Further, the center wavelength affects a lateral resolution of the obtained tomographic images, and hence can be set to as short a wavelength as possible. For this reason, in Embodiment 1, the light source 110 having the center wavelength of 855 nm is used. Specific numerical values of the center wavelength and the wavelength bandwidth of the light source 110 in this specification are merely exemplary, and may be set to other numerical values.

Light emitted from the light source 110 is split to the measurement light and the reference light under a desired branching ratio by the coupler 111. After the light from the light source 110 is split by the coupler 111, the measurement light is guided to the sample optical system 120, and the reference light is guided to the reference optical system 130.

<Sample Optical System 120>

The sample optical system 120, to which the measurement light is guided, includes a collimator lens 121, a focus lens 122, an X galvano scanner 123 and a Y galvano scanner 124, which are variable in angle, and objective lenses 125 and 126. The measurement light is guided as a beam spot onto the fundus Er of the eye to be inspected E through those elements.

The collimator lens 121 is configured to convert the measurement light that has entered the sample optical system 120 into collimated light, and output the collimated light. The focus lens 122 is held by a driving member (not shown), which is controlled by the control unit 200, to be movable in the optical axis direction indicated by the arrow in FIG. 1. The control unit 200 can move the focus lens 122 in the optical axis direction to focus the measurement light on the eye to be inspected E.

The X galvano scanner 123 and the Y galvano scanner 124 can be rotated under control by the control unit 200 to deflect the measurement light in an X-axis direction and a Y-axis direction, respectively. Therefore, the X galvano scanner 123 and the Y galvano scanner 124 can be rotated to scan the beam spot that has been guided onto the fundus Er two-dimensionally over the fundus. The measurement light that has been guided onto the eye to be inspected E and has been reflected and scattered by the fundus Er of the eye to be inspected E passes through the sample optical system 120, and then is guided to the interference optical system 140 through the coupler 111.

When the anterior eye Ea of the eye to be inspected E is to be imaged, the X galvano scanner 123 and the Y galvano scanner 124 can be driven to guide the beam spot of the measurement light onto the anterior eye Ea. Further, in Embodiment 1, the X galvano scanner 123 and the Y galvano scanner 124 are used as scanning devices, but any other deflecting devices may be used as the scanning devices. For example, a MEMS mirror capable of deflecting the measurement light in two-dimensional directions with one mirror can be used. In Embodiment 1, the X galvano scanner 123 is used as a scanning device for main scanning of the measurement light, and the Y galvano scanner 124 is used as a scanning device for sub-scanning of the measurement light. However, the main scanning direction and the sub-scanning direction are not limited to the X-axis direction and the Y-axis direction, respectively. Further, the main scanning direction and the sub-scanning direction may not coincide with the X-axis direction and the Y-axis direction. Therefore, the main scanning direction and the sub-scanning direction may be determined as appropriate depending on a two-dimensional tomographic image or three-dimensional tomographic image that is desired to be captured.

<Reference Optical System 130>

Meanwhile, the reference optical system 130, to which the reference light is guided, includes a polarization control paddle 134, a collimator lens 131, an ND filter 132, and a mirror 133. The polarization control paddle 134 is formed of optical fibers bundled into a plurality of rings, and is capable of controlling a polarization state of the reference light with respect to a polarization state of the measurement light so that an interference state between the measurement light and the reference light is improved.

The collimator lens 131 is configured to convert the reference light that has entered the reference optical system 130 into collimated light, and output the collimated light. The ND filter 132 is configured to attenuate an amount of the incident reference light to a predetermined amount. The mirror 133 is held by a driving member (not shown), which is controlled by the control unit 200, to be movable in the optical axis direction, and can be moved in the optical axis direction to correct a difference in optical path length from the sample optical system 120. The reference light that has passed through the ND filter 132 is reflected by the mirror 133 while maintaining the collimated state, and is bent back to the same optical path. The bent-back reference light passes through the ND filter 132 and the collimator lens 131, and is then guided to the interference optical system 140 through the coupler 111.

<Interference Optical System 140>

The measurement light that has returned from the sample optical system 120 and the reference light that has returned from the reference optical system 130 are coupled by the coupler 111, and are guided as interference light to the interference optical system 140. The interference optical system 140 includes a collimator lens 141, a diffraction grating 142, a lens 143, and a line sensor 144.

The collimator lens 141 is configured to convert the interference light that has entered the interference optical system 140 into collimated light, and output the collimated light. The diffraction grating 142 is configured to disperse the incident interference light. The dispersed light enters the line sensor 144 via the lens 143. The line sensor 144 is configured to output an interference signal (OCT signal) based on the incident light. The line sensor 144 is arranged so that each pixel receives light corresponding to a wavelength component of the light dispersed by the diffraction grating 142.

<Configuration of Control Unit 200>

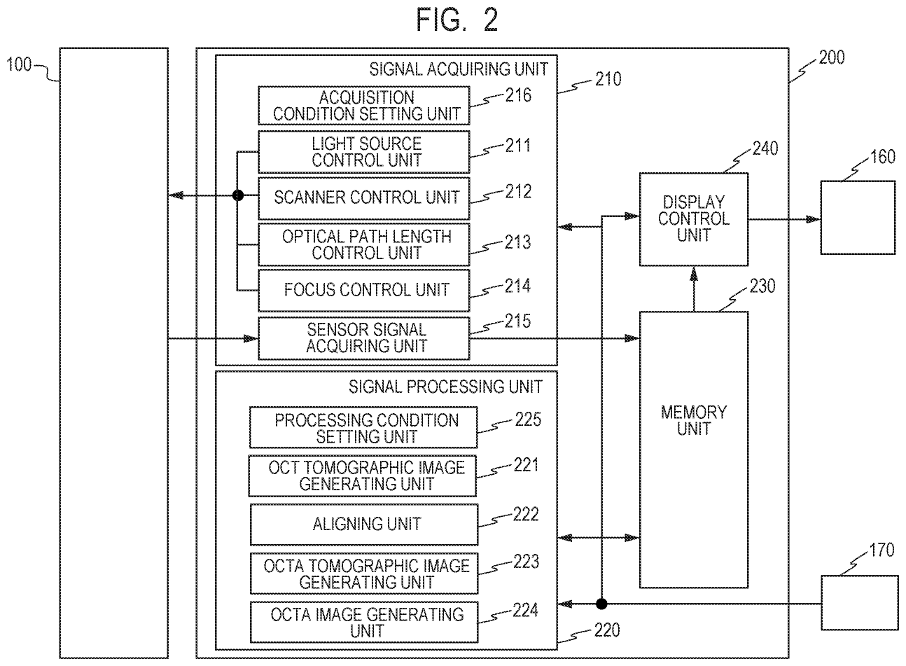

Next, referring to FIG. 2, a schematic configuration of the control unit 200 is described. The control unit 200 includes a signal acquiring unit 210 (acquiring unit), a signal processing unit 220 (image generating unit), a memory unit 230, and a display control unit 240.

The signal acquiring unit 210 includes a light source control unit 211, a scanner control unit 212, an optical path length control unit 213, a focus control unit 214, a sensor signal acquiring unit 215, and an acquisition condition setting unit 216. The signal acquiring unit 210 is connected to the light source 110, the X galvano scanner 123 and the Y galvano scanner 124, the driving members (not shown) configured to drive the mirror 133 and the focus lens 122, and the line sensor 144. The signal acquiring unit 210 is also connected to the operation input unit 170, and is configured to control the light source 110 and other components depending on input contents to scan the measurement light over the anterior eye Ea or the fundus Er of the eye to be inspected E. Then, the signal acquiring unit 210 can acquire the OCT signal obtained by wavelength resolving the interference light of the return light of the measurement light and the reference light from the line sensor 144.

The light source control unit 211 is connected to the light source 110 of the imaging apparatus unit 100, and can perform ON/OFF control and other control on the light source 110. The scanner control unit 212 can control the X galvano scanner 123 and the Y galvano scanner 124 to scan the measurement light over a suitable position on the anterior eye Ea or the fundus Er of the eye to be inspected E.

The optical path length control unit 213 can control a motor or other driving member (not shown) configured to drive the mirror 133 to adjust an optical path length of the reference light in accordance with an optical path length of the measurement light. The focus control unit 214 can control a motor or other driving member (not shown) configured to drive the focus lens 122 to focus the measurement light on the anterior eye Ea or the fundus Er of the eye to be inspected E. The sensor signal acquiring unit 215 can acquire the OCT signal input from the line sensor 144, and store the OCT signal in the memory unit 230. The signal acquiring unit 210 can also send the OCT signal acquired by the sensor signal acquiring unit 215 to the signal processing unit 220.

The acquisition condition setting unit 216 is configured to set conditions for acquiring the OCT signal depending on a scan pattern, a scan size, or a scan position relating to imaging, the imaging mode for capturing a moving image or a still image, a frame rate of the moving image, and the like. The signal acquiring unit 210 can control the light source 110, the X galvano scanner 123, the Y galvano scanner 124, and other components in accordance with the OCT signal acquisition conditions set by the acquisition condition setting unit 216 to acquire the OCT signal having a desired data amount.

The signal processing unit 220 includes an OCT tomographic image generating unit 221, an aligning unit 222, an OCTA tomographic image generating unit 223, an OCTA image generating unit 224, and a processing condition setting unit 225. Further, the signal processing unit 220 is connected to the operation input unit 170, and can read signal data in accordance with contents input by operations to generate OCT tomographic images, OCTA tomographic images, and OCTA images.

The OCT tomographic image generating unit 221 is configured to perform frequency analysis using fast Fourier transform (FFT) on the OCT signal acquired by the signal acquiring unit 210 or the memory unit 230 to generate OCT data obtained by converting tomographic information into luminance values or density values. The OCT tomographic image generating unit 221 is configured to generate tomographic images of the eye to be inspected E based on the generated OCT data. The OCT tomographic image generating unit 221 may acquire the OCT data, for example, the luminance values from the signal acquiring unit 210 or the memory unit 230 to generate the tomographic images based on the acquired OCT data. Further, as a method of generating the OCT data and a method of generating the OCT tomographic images, any known method may be used. The interference signal, the Fourier-transformed signal generated based on the interference signal, a signal obtained by performing any processing on the Fourier-transformed signal, the OCT data, which is luminance data of the tomographic images generated by the OCT tomographic image generating unit 221, and other such data are hereinafter collectively referred to as “tomographic data”.

The aligning unit 222 can align a plurality of OCT tomographic images captured of substantially the same part of the eye to be inspected with the use of characteristic points or the like in the images. The OCTA tomographic image generating unit 223 is configured to generate motion contrast data based on the aligned OCT tomographic images to generate the OCTA tomographic images. As a method of generating the motion contrast data, any known method may be used. For example, the OCTA tomographic image generating unit 223 can determine, as the motion contrast data, a decorrelation value, a variance, or a value (maximum value/minimum value) obtained by dividing a maximum value by a minimum value of pixel values at corresponding pixels in the aligned OCT tomographic images.

The OCTA image generating unit 224 is configured to project three-dimensional OCTA volume data based on the OCTA tomographic images in tomographic in-plane directions (axial direction of sub-scanning and axial direction of main scanning) to generate the OCTA images. In Embodiment 1, the OCTA image generating unit 224 takes, at each pixel location of a plane corresponding to a front surface of the eye to be inspected E, an average value of the motion contrast data in a desired depth range as a representative value, and uses the representative value to determine a pixel value at each pixel location, to thereby generate the OCTA images. The representative value is not limited to the average value of the motion contrast data, and may be a median, a mode, or a maximum value, for example.

The processing condition setting unit 225 is configured to set conditions for processing the OCT signal depending on the scan pattern, the scan size, or the scan position relating to imaging, the imaging mode for capturing a moving image or a still image, and the like. The signal processing unit 220 can adjust, of the acquired OCT signal, a data amount of the OCT signal used in the processing of generating the OCT tomographic images, the OCTA tomographic images, or the OCTA images in accordance with the OCT signal processing conditions set by the processing condition setting unit 225.

The memory unit 230 is connected to the signal acquiring unit 210, the signal processing unit 220, and the display control unit 240, and can store the patient information, the OCT signal, the OCT tomographic images, the OCTA tomographic images, and the OCTA images of the eye to be inspected E, and other data. The display control unit 240 is connected to the memory unit 230 and the display unit 160, and can display the patient information, various images, and other information on the display unit 160. The signal acquiring unit 210, the signal processing unit 220, and the display control unit 240 may be formed of software modules to be executed by a CPU or a MPU of the control unit 200, or of a circuit configured to achieve a particular function, for example, an ASIC. Further, the memory unit 230 can be formed with the use of any memory, optical disc, or other storage medium.

<Display Contents of Display Unit 160>

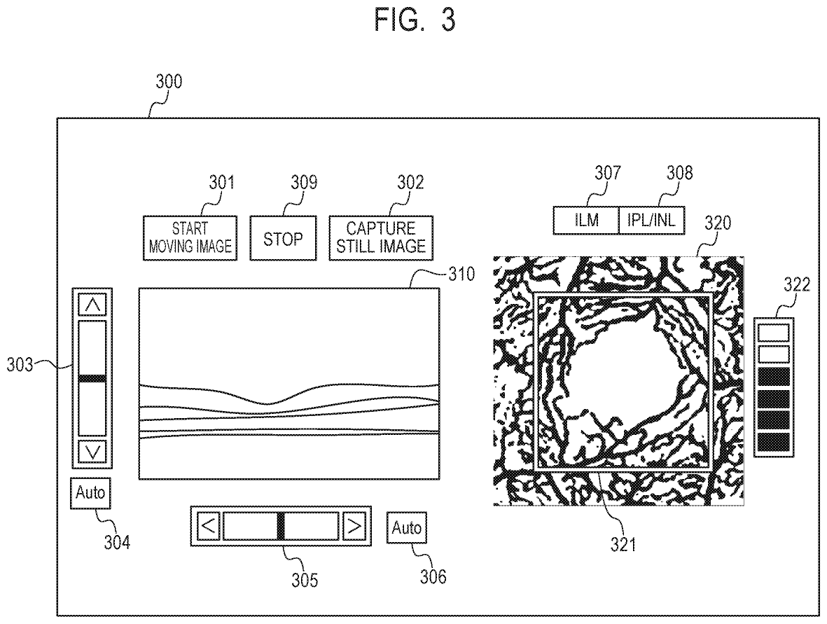

On the display unit 160, a screen 300 of FIG. 3 is displayed. On the screen 300, an OCT tomographic image 310 and an OCTA image 320 are displayed. The screen 300 also includes a “start moving image” button 301, a stop button 309, a “capture still image” button 302, slide bars 303 and 305, and “Auto” buttons 304 and 306. The screen 300 further includes pulldown menus 307 and 308 for selecting an OCTA extraction range, an imaging range frame 321, and an indicator 322.

In regions of the OCT tomographic image 310 and the OCTA image 320, images stored in the memory unit 230 are displayed in accordance with control by the display control unit 240. Each of the OCT tomographic image 310 and the OCTA image 320 may be displayed as a moving image, or as a still image.

The “start moving image” button 301 is a button used to issue an instruction to start capturing a moving image. The “capture still image” button 302 is a button used to issue an instruction to start capturing a still image. When the examiner uses the operation input unit 170 to click the “capture still image” button 302 while a preview image (moving image) is displayed, the examiner can issue an instruction to capture a still image under desired conditions based on the preview image.

The slide bar 303 is linked to a position of the focus lens 122, and when the examiner uses the operation input unit 170 to operate the slide bar 303, the focus of the measurement light can be adjusted. Further, the “Auto” button 304 is a button used to issue an instruction to automatically adjust the focus. When the “Auto” button 304 is clicked, the control unit 200 automatically adjusts the focus of the measurement light based on at least one of the OCT tomographic image, the OCTA tomographic image, or the OCTA image.

The slide bar 305 is linked to a position of the mirror 133, and when the examiner uses the operation input unit 170 to operate the slide bar 305, the optical path length of the reference light can be adjusted. Further, the “Auto” button 306 is a button used to issue an instruction to automatically adjust the optical path length. When the “Auto” button 306 is clicked, the control unit 200 automatically adjusts the optical path length based on at least one of the OCT tomographic image, the OCTA tomographic image, or the OCTA image.

The pulldown menus 307 and 308 for selecting the OCTA extraction range are used to select a layer range of a retina of the fundus Er that is desired to be extracted as an OCTA image. The OCTA image generating unit 224 is configured to determine the representative value of the motion contrast data for the layer range (depth range) of the retina layer designated with the use of the pulldown menus 307 and 308 for selecting the OCTA extraction range to determine the pixel value of an OCTA image, and generate the OCTA image. The stop button 309 is a button used to stop imaging, and when the examiner uses the operation input unit 170 to click the stop button 309, the imaging that is being executed is stopped.

The imaging range frame 321 is used to select a scan range during imaging. When the examiner uses the operation input unit 170 to adjust the imaging range frame 321 superimposed on the OCTA image 320 (including the preview image), the examiner can set the scan position and the size for imaging.

The indicator 322 is an index indicating image quality based on a focus state of an image and other factors, and the examiner can check a state of imaging adjustment based on indication on the indicator 322. The control unit 200 can determine the image quality based on at least one of the OCT tomographic image, the OCTA tomographic image, or the OCTA image to determine the indication on the indicator 322. Any other buttons and images may be displayed on the screen 300.

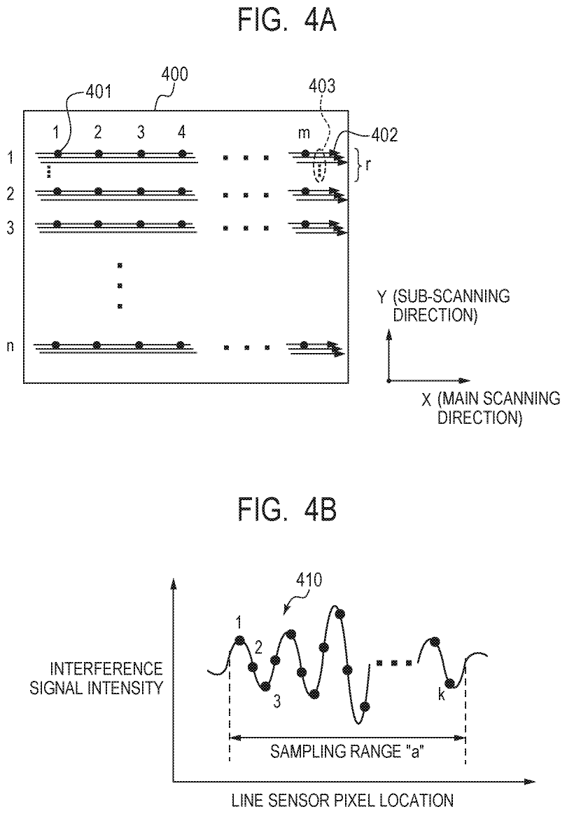

Next, referring to FIG. 4A and FIG. 4B, signal acquisition conditions relating to OCTA imaging are described.

<OCT Signal Acquisition Conditions>

First, OCT signal acquisition conditions are described. In FIG. 4A, in a scan region 400 corresponding to the fundus Er of the eye to be inspected E, the scan pattern in which the X galvano scanner 123 and the Y galvano scanner 124 are controlled to scan the fundus Er is illustrated.

In this specification, using the measurement light to acquire a signal in a depth direction at one point on the fundus Er is called an “A-scan”, and is indicated by a point 401 on FIG. 4A. Further, a series of A-scans performed in a period in which at least one of the X galvano scanner 123 or the Y galvano scanner 124 is controlled to be driven to perform one scan with the measurement light in the main scanning direction is called a “B-scan”, and is indicated by an arrow 402. Still further, a set of B-scans repeated along substantially the same locus (at substantially the same part) for OCTA imaging is called a “set of B-scans”, and is indicated by a broken line 403.

The acquisition condition setting unit 216 of the signal acquiring unit 210 can set the number “m” of A-scans acquired in one B-scan. Further, the acquisition condition setting unit 216 can set the number “n” of sets of B-scans, which is the number of movements of the scanning locus in the sub-scanning direction included in the scan pattern. As the number “m” of A-scans and the number “n” of sets of B-scans become larger, lateral resolution of the OCTA image can be increased.

Further, the acquisition condition setting unit 216 can set the number “r” of repetitions, which is the number of B-scans repeated along substantially the same locus. As the number “r” of repetitions becomes larger, random noise can be reduced, and contrast of the OCTA image can be increased when the OCTA tomographic image is generated.

In FIG. 4B, an interference signal waveform 410 input to the line sensor 144 is illustrated, in which the horizontal axis indicates a line sensor pixel location, and the vertical axis indicates an interference signal intensity. The acquisition condition setting unit 216 can set a sampling number “k” corresponding to the number of pixels from which the signal is to be read of line sensor pixels. As the sampling number “k” becomes larger, the depth range of the OCT tomographic image can be increased.

Further, the acquisition condition setting unit 216 can set a sampling range “a” corresponding to a range of pixels from which the signal is to be read of the line sensor pixels. As the sampling range “a” becomes wider, the resolution in the depth direction of the OCT tomographic image can be increased.

The configuration in which raster scan is performed in the two-dimensional directions as the scan pattern has been described above, but the scan pattern is not limited thereto. Any scan pattern, for example, a cross scan in which scanning loci are formed of two straight lines that are orthogonal to each other, a circle scan in which a scanning locus is substantially circular, a radial scan, or other scans may be used.

The acquisition condition setting unit 216 can set values of various conditions including the number “m” of A-scans, the number “n” of sets of B-scans, the number “r” of repetitions, the sampling number “k”, and the sampling range “a”, which have been described above, based on the scan pattern, the scan size, or the scan position. In this case, for example, the acquisition condition setting unit 216 can refer to a table in which the scan pattern and various conditions are associated with each other, and set values of associated parameters as the OCT signal acquisition conditions based on the scan pattern, for example.

Further, in Embodiment 1, the acquisition condition setting unit 216 can change the values of those conditions for a case where the OCTA images are to be captured as a moving image and a case where the OCTA images are to be captured as a still image. As the values of the conditions become larger, the image quality of the OCTA images is increased or the imaging range is enlarged, but the data amount of the acquired OCT signal is increased. In contrast, as the values of the conditions become smaller, the image quality of the OCTA images is reduced or the imaging range is reduced, but the data amount of the acquired OCT signal is reduced.

In view of the above-mentioned circumstances, when a moving image is to be captured, the values of the OCT signal acquisition conditions are reduced to reduce the data amount of the acquired OCT signal, to thereby reduce the data amount used in generating the OCTA images. As a result, computational complexity required for the processing of generating the OCTA images can be reduced, and hence the generation of the moving image of the OCTA images can be sped up.

In view of the above-mentioned relationship, the acquisition condition setting unit 216 can set the above-mentioned various conditions based on the frame rate of the moving image. For example, when the frame rate is set high in real-time display, it is required to generate the OCTA images at a higher speed. Therefore, through reducing the values of the OCT signal acquisition conditions to reduce the data used in generating the OCTA images, the time required to generate the OCTA images can be reduced, and a high frame rate can be achieved.

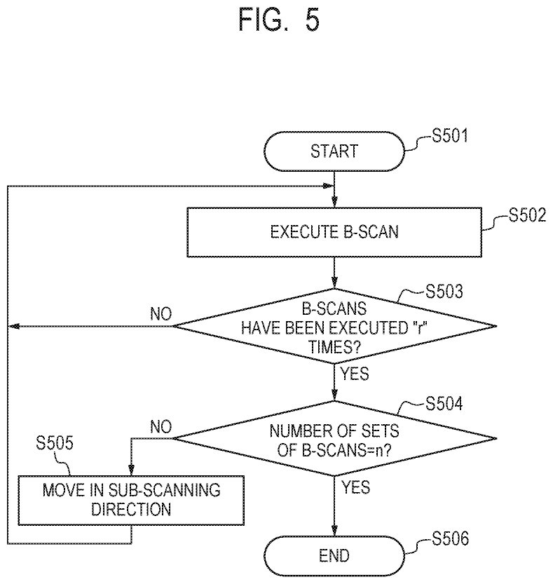

Now, referring to FIG. 5 to FIG. 7, an OCT signal acquiring sequence, an OCTA signal processing sequence, and an imaging sequence are described. First, referring to FIG. 5, the OCT signal acquiring sequence is described. FIG. 5 is a flow chart of the OCT signal acquiring sequence.

<OCT Signal Acquiring Sequence>

After the OCTA signal processing sequence is started in Step S501, the processing proceeds to Step S502.

In Step S502, the light source control unit 211 turns on the light source 110. Then, the scanner control unit 212 controls to drive at least one of the X galvano scanner 123 or the Y galvano scanner 124 to perform a B-scan including “m” A-scans with the measurement light based on the light from the light source 110. The sensor signal acquiring unit 215 samples the OCT signal input from the line sensor 144, and stores the sampled OCT signal in the memory unit 230.

In Step S503, the signal acquiring unit 210 determines whether a B-scan has been performed “r” times along substantially the same scanning locus (at substantially the same part). When the signal acquiring unit 210 determines that the B-scan has been performed “r” times, the processing proceeds to Step S504. In contrast, when the signal acquiring unit 210 determines that the B-scan has not been performed “r” times, the processing returns to Step S502, in which the signal acquiring unit 210 performs a B-scan along substantially the same scanning locus.

In Step S504, the signal acquiring unit 210 determines whether the sets of B-scans have been performed “n” times. When the signal acquiring unit 210 determines that the sets of B-scans have not been performed “n” times, the processing proceeds to Step S505. In Step S505, the scanner control unit 212 moves the galvano scanner in the sub-scanning direction, and the processing returns to Step S502, in which a B-scan at a different sub-scanning position is performed.

When the signal acquiring unit 210 determines in Step S504 that the sets of B-scans have been performed “n” times, the processing proceeds to Step S506, in which the OCT signal acquiring sequence is ended.

<OCTA Signal Processing Sequence>

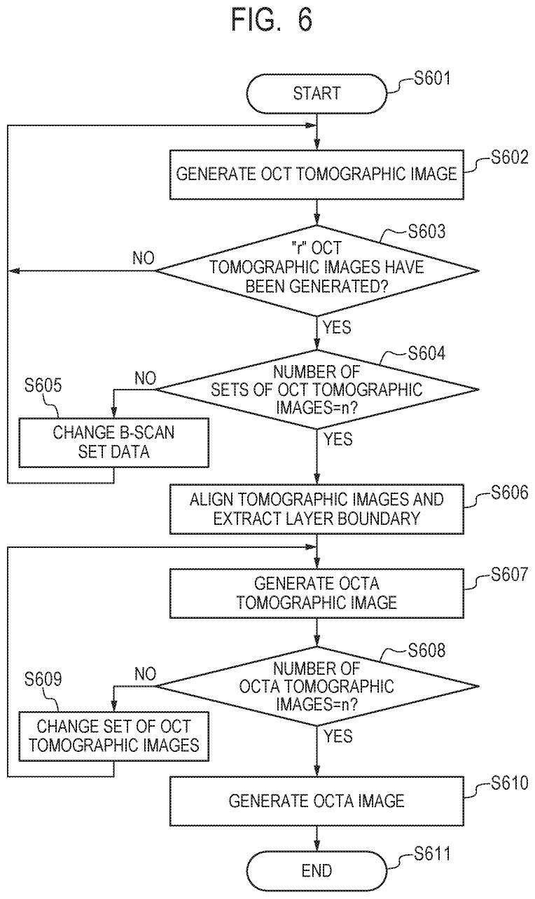

Next, referring to FIG. 6, the OCTA signal processing sequence is described. FIG. 6 is a flow chart of the OCTA signal processing sequence. After OCTA signal processing is started in Step S601, the processing proceeds to Step S602.

In Step S602, the OCT tomographic image generating unit 221 reads the OCT signal for one B-scan from the memory unit 230, and performs the frequency analysis by FFT or the like to generate an OCT tomographic image. The OCT tomographic image generating unit 221 stores the generated image in the memory unit 230.

In Step S603, the signal processing unit 220 determines whether “r” OCT tomographic images have been generated for data (B-scan data) acquired in “r” B-scans performed along substantially the same scanning locus. When the signal processing unit 220 determines that “r” OCT tomographic images have been generated, the processing proceeds to Step S604. In contrast, when the signal processing unit 220 determines that “r” OCT tomographic images have not been generated, the processing returns to Step S602, in which the OCT tomographic image generating unit 221 reads another OCT signal along substantially the same locus to generate an OCT tomographic image.

In Step S604, the signal processing unit 220 determines whether OCT tomographic images have been generated for data (B-scan set data) acquired in “n” sets of B-scans to generate “n” sets of OCT tomographic images. When the signal processing unit 220 determines that “n” sets of OCT tomographic images have not been generated, the processing proceeds to Step S605. In Step S605, the signal processing unit 220 changes B-scan set data to be subjected to signal processing, and the processing returns to Step S602, in which the OCT tomographic image generating unit 221 generates an OCT tomographic image based on a different piece of B-scan set data.

When the signal processing unit 220 determines in Step S604 that “n” sets of OCT tomographic images have been generated, the processing proceeds to Step S606. In Step S606, the aligning unit 222 first reads the OCT tomographic images in units of a set of B-scans from the memory unit 230, and aligns “r” OCT tomographic images included in one set of B-scans.

Specifically, the aligning unit 222 first selects any one of “r” OCT tomographic images as a template. For example, the aligning unit 222 can select a tomographic image that has been generated first as a tomographic image to be selected as the template. Alternatively, the aligning unit 222 may calculate a correlation value for all the combinations of “r” OCT tomographic images to determine a sum of correlation coefficients for each frame, and select a tomographic image for which the sum of the correlation coefficients is at the maximum, as the template.

The aligning unit 222 then compares each OCT tomographic image against the template to determine a displacement amount (δX, δZ, δθ) for each OCT tomographic image, where δX denotes a displacement amount in an X direction (main scanning direction), δZ denotes a displacement amount in a Z direction (depth direction), and δθ denotes a rotational displacement amount. Specifically, the aligning unit 222 calculates a normalized cross-correlation (NCC), which is an index of similarity of the template to the tomographic image of each frame, while changing the position and angle of the template. The aligning unit 222 determines, as the displacement amount, the difference in position between the template and the OCT tomographic image to be compared at the time when the calculated NCC is at the maximum. The index of similarity between the images can be any measure of similarity of characteristics between the template and the OCT tomographic image of the frame to be compared, and any of various indexes serving as such a measure can be used.

The aligning unit 222 aligns the OCT tomographic images by applying a positional correction based on the determined displacement amount (δX, δZ, δθ) to r−1 OCT tomographic images excluding the template. As a result of “r” OCT tomographic images being aligned with each other, pixels at the same coordinates (pixel location) in the images represent the same part of the fundus Er in those pixels. A method of aligning the OCT tomographic images by the aligning unit 222 is not limited to the above-mentioned method, and may be any known method.

After the OCT tomographic images are aligned, the signal processing unit 220 performs a segmentation processing on the OCT tomographic image selected as the above-mentioned template to extract a boundary of a layered structure of the fundus, which is the subject to be inspected. The layer boundaries can be extracted using any known layer boundary extraction technique, as far as the technique can extract an anatomical layer boundary of the fundus. The layer boundary extraction is not limited to the configuration of being performed in Step S606, but may be performed after the OCT tomographic images are generated and before the OCTA images are generated.

In Step S607, the OCTA tomographic image generating unit 223 calculates an amount of change in luminance value (motion contrast data) based on “r” OCT tomographic images acquired in one set of B-scans. The motion contrast data can be calculated through use of any known method as described above. In Embodiment 1, the OCTA tomographic image generating unit 223 determines a decorrelation value of the luminance values of corresponding pixels of two OCT tomographic images to calculate the amount of change in luminance value.

Thereafter, the OCTA tomographic image generating unit 223 converts the amount of change in luminance value of the OCT tomographic images into luminance values, for example, for imaging to generate the OCTA tomographic images. When “r” is 3 or more, the OCTA tomographic image generating unit 223 can average the OCTA tomographic images acquired from two OCT tomographic images based on the OCT signal acquired in a predetermined time interval to generate an averaged OCTA tomographic image. In this case, the OCTA tomographic image generating unit 223 can generate a high-contrast OCTA tomographic image from which random noise is reduced through averaging. The OCTA tomographic image generating unit 223 stores the generated OCTA tomographic image in the memory unit 230.

In Step S608, the signal processing unit 220 determines whether OCTA tomographic images have been generated for “n” sets of OCT tomographic images to generate “n” OCTA tomographic images. When the signal processing unit 220 determines that “n” OCTA tomographic images have not been generated, the processing proceeds to Step S609. In Step S609, the signal processing unit 220 changes the set of OCT tomographic images (or B-scan set data) to be subjected to signal processing. Thereafter, the processing returns to Step S607, in which the OCTA tomographic image generating unit 223 generates an OCTA tomographic image based on a different set of OCT tomographic images.

When the signal processing unit 220 determines in Step S608 that “n” OCTA tomographic images have been generated, the processing proceeds to Step S610. In Step S610, the OCTA image generating unit 224 produces the three-dimensional OCTA volume data based on the “n” OCTA tomographic images generated in Step S607. Then, the OCTA image generating unit 224 recognizes a layer boundary of the fundus retina from the three-dimensional OCTA volume data based on the layer boundary extracted in Step S606. Thereafter, the OCTA image generating unit 224 generates, as the OCTA image, a two-dimensional plane image in the tomographic in-plane directions (axial direction of main scanning and axial direction of sub-scanning) including a desired layer based on the three-dimensional OCTA volume data. The OCTA image generating unit 224 stores the generated OCTA image in the memory unit 230. Thereafter, the processing proceeds to Step S611, in which the OCTA signal processing sequence is ended.

<Imaging Sequence>

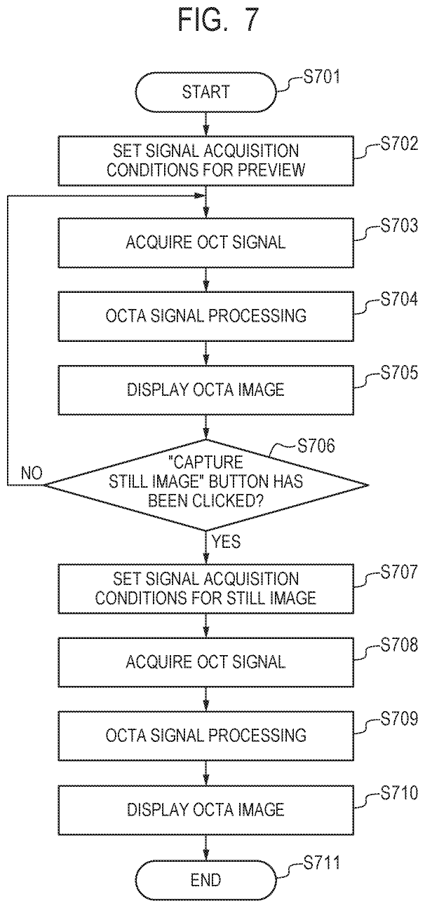

Next, referring to FIG. 7, the imaging sequence in Embodiment 1 is described. FIG. 7 is a flow chart of the imaging sequence in Embodiment 1. First, in Step S701, the control unit 200 detects that the examiner has used the operation input unit 170 to click the “start moving image” button 301 on the screen 300, and starts imaging.

In Step S702, the acquisition condition setting unit 216 sets the OCT signal acquisition conditions for the preview image (moving image). At this time, the acquisition condition setting unit 216 sets the conditions such as the number “m” of A-scans so that an amount of acquired data of the OCT signal for the preview image becomes smaller than an amount of acquired data of the OCT signal for the still image. In reducing the values of the conditions, the number “m” of A-scans and the number “n” of sets of B-scans may be reduced so that the scan range is reduced, or the number “m” of A-scans and the number “n” of sets of B-scans may be reduced by decimation without changing the scan range.

When there is a blank region in which no retina image is captured in the depth direction in the OCT tomographic image, the sampling number “k” can be reduced so as to omit the region. When the sampling number “k” is reduced, the depth range of the OCT tomographic image is narrowed, and hence the position of the mirror 133 may be adjusted in accordance with a position of the retina image to adjust the optical path length of the reference light. Further, when there are two OCT tomographic images at minimum along substantially the same scanning locus (at substantially the same part), an OCTA image can be generated. Therefore, the number “r” of repetitions can be reduced to 2.

In Step S703, the signal acquiring unit 210 acquires the OCT signal in accordance with the set acquisition conditions and the above-mentioned OCT signal acquiring sequence. In Step S704, the signal processing unit 220 performs the OCTA signal processing in accordance with the above-mentioned OCTA signal processing sequence to generate the OCTA image. In Step S705, the display control unit 240 reads the image data from the memory unit 230, and displays the OCT tomographic image 310 and the OCTA image 320 on the screen 300.

In Step S706, the control unit 200 detects whether the examiner has clicked the “capture still image” button 302 on the screen 300 with the use of the operation input unit 170. When the control unit 200 detects that the “capture still image” button 302 has been clicked, the processing proceeds to Step S707. In contrast, while the control unit 200 does not detect that the “capture still image” button 302 has been clicked, Step S703 to Step S705 are repeatedly executed to display the OCT tomographic image 310 and the OCTA image 320 as moving images.

While the moving images are displayed, the examiner can perform the imaging adjustment with the use of various buttons and slide bars displayed on the screen 300. For example, the examiner can select the layer range of the fundus retina that is desired to be extracted as an OCTA image with the pulldown menus 307 and 308 for selecting the OCTA extraction range while checking the OCT tomographic image 310. Further, in the screen 300 illustrated in FIG. 3, only one OCTA image 320 is displayed. However, a plurality of layer ranges of the fundus retina may be selected, and a plurality of OCTA images may be accordingly displayed at the same time. Further, the examiner can select the scan range in capturing the still image with the use of the imaging range frame 321 while checking the OCTA image 320.

In Step S707, the acquisition condition setting unit 216 sets the OCT signal acquisition conditions for a still image. At this time, the acquisition condition setting unit 216 sets the OCT signal acquisition conditions such as the number “m” of A-scans so that the amount of acquired data of the OCT signal for the still image becomes larger than the amount of acquired data of the OCT signal for the moving image.

In Step S708, the signal acquiring unit 210 acquires the OCT signal in accordance with the set acquisition conditions and the above-mentioned OCT signal acquiring sequence. In Step S709, the signal processing unit 220 performs the OCTA signal processing in accordance with the above-mentioned OCTA signal processing sequence to generate the OCTA image. In Step S710, the display control unit 240 reads the image data from the memory unit 230, and displays the OCT tomographic image 310 and the OCTA image 320 as still images on the screen 300. Thereafter, the processing proceeds to Step S711, in which the OCTA imaging sequence is ended.

In Embodiment 1, as described above, the OCT signal acquisition conditions for the moving image and for the still image are changed in Step S702 and Step S707 so that the amount of acquired data of the OCT signal for the moving image becomes smaller than the acquired data of the OCT signal for the still image. When the amount of acquired data of the OCT signal is large, the OCTA image of high quality or a wide range can be generated. In contrast, when the amount of acquired data of the OCT signal is small, the OCTA image can be generated in a short period of time.

Therefore, in Step S702, in order to display the moving image, the conditions are set so that the data amount of the OCT signal acquired in Step S703 becomes smaller than the data amount of the OCT signal acquired in Step S708. As a result, the time required for the OCTA signal processing in Step S704 for generating the OCTA image corresponding to one frame of the moving image can be reduced, generation of the moving image can be sped up, and the frame rate in displaying the moving image can be increased.

For example, it takes about 3 seconds to generate one OCTA image with the number “m” of A-scans being set to 256 and the number “n” of sets of B-scans being set to 256, that is, to generate one 256×256 pixel×pixel OCTA image. Then, when the number “m” of A-scans is set to 64 and the number “n” of sets of B-scans is set to 64 to reduce the data amount of the OCT signal, the required signal processing time is reduced to one-sixteenth, and one OCTA image can be generated in about 0.2 second. Therefore, generation and display of the moving image of the OCTA images can be sped up.

When the amount of acquired data of the OCT signal is decimated, the number of pixels of the OCTA images is reduced, and hence the image quality is reduced. To address this problem, known data acquisition and display methods such as interlacing can be used to suppress the reduction of image quality.

As described above, the control unit 200 in Embodiment 1 includes the signal acquiring unit 210 and the signal processing unit 220. The signal acquiring unit 210 acquires a plurality of pieces of tomographic data indicating tomographic information at substantially the same position of the subject to be inspected. The signal processing unit 220 generates the OCTA images using the plurality of pieces of tomographic data. Further, in the control unit 200, the data amount of the tomographic data used in generating one OCTA image when the OCTA images are to be generated as a moving image is smaller than the data amount of the tomographic data used in generating one OCTA image when the OCTA image is to be generated as a still image.

In particular, the data amount of the tomographic data acquired by the signal acquiring unit 210 to generate one OCTA image when the moving image is to be generated is smaller than the data amount of the tomographic data acquired to generate one OCTA image when the still image is to be generated. More specifically, the signal acquiring unit 210 sets at least one value of the OCT signal acquisition conditions in acquiring the tomographic data to generate one OCTA image when the moving image is to be generated smaller than the value of the OCT signal acquisition conditions when the still image is to be generated. Here, the OCT signal acquisition conditions include the number of A-scans, the number of sets of B-scans, the number of repetitions of scans at substantially the same position, the sampling number of the interference signal, and the sampling range of the interference signal.

In the control unit 200 in Embodiment 1, the amount of acquired data of the OCT signal for capturing the moving image becomes smaller than the amount of acquired data of the OCT signal for capturing the still image, with the result that the computational complexity and time involved in the generation of the OCTA images can be reduced, and the generation of the moving image of the OCTA images can be sped up. Further, in Embodiment 1, the amount of acquired data of the OCT signal is reduced, and hence the time involved in acquiring the OCT signal can also be reduced. Therefore, also in this respect, the time from the acquisition of the OCT signal to the generation of the moving image can be reduced, and the generation and display of the moving image can be sped up.

In Embodiment 1, OCT signal processing conditions to be described later are not set, and hence the signal processing unit 220 may not include the processing condition setting unit 225.

### Embodiment 2

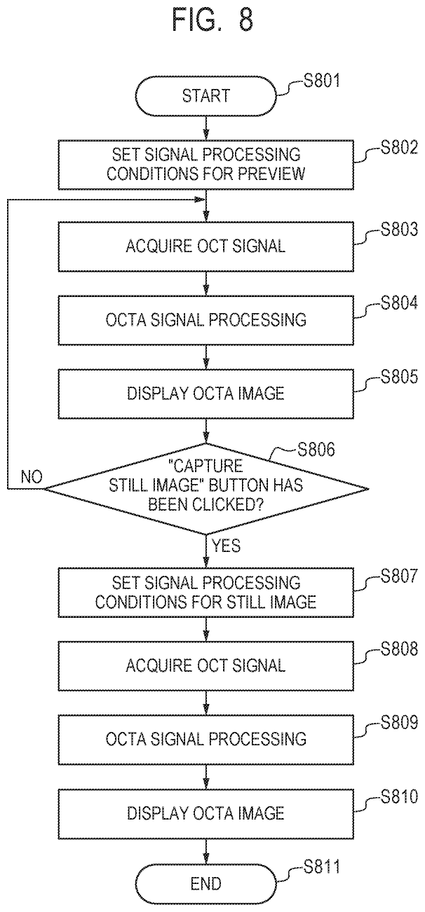

In Embodiment 1, the OCT signal acquisition conditions are changed for the generation of the moving image of the OCTA images and for the generation of the still image. In contrast, in Embodiment 2 of the present invention, the OCT signal processing conditions used in generating the OCTA images are changed for the generation of the moving image of the OCTA images and for the generation of the still image to speed up the generation of the moving image. Now, referring to FIG. 8, the control unit in Embodiment 2 is described. A configuration of the OCT apparatus in Embodiment 2 is similar to the OCT apparatus in Embodiment 1. Therefore, the same reference symbols are used, and a description thereof is omitted. Now, the control unit 200 in Embodiment 2 is described mainly for differences from Embodiment 1.

In the control unit 200 in Embodiment 2, instead of changing the OCT signal acquisition conditions for the generation of the moving image of the OCTA images and for the generation of the still image, the processing condition setting unit 225 of the signal processing unit 220 changes an amount of processed data of the OCT signal used in generating the OCTA images. In particular, the processing condition setting unit 225 sets the OCT signal processing conditions for the generation of the moving image and for the generation of the still image so that the amount of processed data of the OCT signal in generating the moving image becomes smaller than the amount of processed data of the OCT signal in generating the still image. Thereafter, the OCT tomographic image generating unit 221 and other components use, of the acquired OCT signal, the OCT signal corresponding to the set processing conditions to generate the OCT tomographic images, to thereby reduce the computational complexity and time involved in generating the OCTA images, and speed up the generation of the moving image of the OCTA images.

<OCT Signal Processing Conditions>

Now, the OCT signal processing conditions are described. In Embodiment 2, the processing condition setting unit 225 sets at least one of the number “m” of A-scans, the number “n” of sets of B-scans, the number “r” of repetitions, the sampling number “k”, or the sampling range “a” as processing conditions for the OCT signal used for the OCTA images. As described above, the data amount involved in generating the OCT tomographic images and the OCTA images is changed depending on the number “m” of A-scans, the number “n” of sets of B-scans, the number “r” of repetitions, the sampling number “k”, and the sampling range “a”. Therefore, of the acquired OCT signal, the OCT signal corresponding to the set processing conditions can be used to change the data amount involved in generating the OCT tomographic images and the OCTA images.

As with the OCT signal acquisition conditions, the OCT signal processing conditions may be set based on the scan pattern, the scan size, the scan position, and the imaging mode, for example. Further, as with the OCT signal acquisition conditions, the OCT signal processing conditions can also be set depending on the frame rate of the moving image.

<Imaging Sequence>

Next, referring to FIG. 8, the imaging sequence in Embodiment 2 is described. FIG. 8 is a flow chart of the imaging sequence in Embodiment 2. First, in Step S801 of FIG. 8, the control unit 200 detects that the examiner has clicked the “start moving image” button 301 on the screen 300 with the use of the operation input unit 170, and starts imaging.

In Step S802, the processing condition setting unit 225 sets OCT signal processing conditions for the preview image (moving image). Here, the processing condition setting unit 225 sets the conditions such as the number “m” of A-scans so that the amount of processed data of the OCT signal for the preview image becomes smaller than the amount of processed data of the OCT signal for the still image. The setting of the values of the conditions is similar to Step S702 in Embodiment 1, and hence a description thereof is omitted. As a difference from Embodiment 1, the values may be evenly reduced or decimated, or a proportion of the number to be reduced or decimated in a center portion of the B-scan image and that of the number to be reduced or decimated in a peripheral portion may be different (the number in the peripheral portion may be set larger).

In Step S803, the signal acquiring unit 210 acquires the OCT signal in accordance with the above-mentioned OCT signal acquiring sequence. In Step S804, the signal processing unit 220 performs the OCTA signal processing in accordance with the set processing conditions and the above-mentioned OCTA signal processing sequence to generate the OCTA images. More specifically, the signal processing unit 220 uses, of the acquired OCT signal, the OCT signal matching the set processing conditions to perform the OCTA signal processing in accordance with the OCTA signal processing sequence. The processing in Step S805 and Step S806 is similar to Steps S705 and S706 in Embodiment 1, and hence a description thereof is omitted.

In Step S807, the processing condition setting unit 225 sets OCT signal processing conditions for a still image. Here, the processing condition setting unit 225 sets OCT signal processing conditions such as the number “m” of A-scans so that the amount of processed data of the OCT signal for the still image becomes larger than the amount of processed data of the OCT signal for the moving image.

In Step S808, the signal acquiring unit 210 acquires the OCT signal in accordance with the above-mentioned OCT signal acquiring sequence. In Step S809, the signal processing unit 220 performs the OCTA signal processing in accordance with the set processing conditions and the above-mentioned OCTA signal processing sequence to generate the OCTA images. The subsequent processing is similar to Steps S710 and S711 in Embodiment 1, and hence a description thereof is omitted.

As described above, in Embodiment 2, the signal processing unit 220 uses, as the tomographic data processed to generate one OCTA image when the moving image is to be generated, a part of the tomographic data acquired by the signal acquiring unit 210 to generate one motion contrast image. For example, when the moving image is to be generated, the signal processing unit 220 uses data obtained by decimating the plurality of pieces of tomographic data acquired by the signal acquiring unit 210 to generate one OCTA image.

More specifically, when the moving image is to be generated, the signal processing unit 220 uses tomographic data based on values obtained by reducing at least one value of the signal processing conditions of the tomographic data acquired by the signal acquiring unit 210 to generate one OCTA image, to generate one OCTA image. Here, the signal processing conditions include the number of A-scans, the number of sets of B-scans, the number of repetitions of scans at substantially the same position, the sampling number of the interference signal, and the sampling range of the interference signal. In such a configuration, the amount of processed data of the OCT signal for capturing the moving image becomes smaller than the amount of processed data of the OCT signal for capturing the still image, with the result that the computational complexity and time involved in generating the OCTA images can be reduced, and the generation of the moving image of the OCTA images can be sped up.

In Embodiment 2, when the preview image is to be generated, the OCTA images are generated using the OCT signal, of the acquired OCT signal, based on the processing conditions set so that the amount of processed data thereof becomes smaller. Therefore, after the imaging sequence is ended, OCTA images of high quality may be generated based on the acquired OCT signal for which the data amount is not reduced to display the moving image.

In displaying the preview image, when the time required for the signal processing for an OCTA image of one frame becomes longer than the time required for the signal acquisition, display of OCTA images does not catch up with imaging. In this case, display of images at substantially the same time as imaging, that is, real-time display cannot be performed, and observation becomes difficult. Therefore, in Step S802, the processing condition setting unit 225 sets the data amount used in the signal processing to be reduced so that, as the signal processing conditions for preview, the signal processing time becomes shorter than the signal acquisition time.

In other words, when the moving image is to be generated, the signal processing unit 220 reduces the data amount used in generating the OCTA image so that the time required to process the tomographic data to generate one OCTA image becomes shorter than the time required to acquire the tomographic data to generate one OCTA image. The processing condition setting unit 225 may set the data amount used in the signal processing to be reduced so that the time required for the signal processing falls within the time set for displaying an OCTA image of one frame.

Further, in Embodiment 2, the signal acquisition conditions are not set, and hence the signal acquiring unit 210 may not include the acquisition condition setting unit 216.

### Embodiment 3

In Embodiment 3 of the present invention, a moving image is captured separately from the display of the preview image, and acquisition conditions and processing conditions are set so that the amount of acquired data and the amount of processed data of the OCT signal are different for generation of a preview image, generation of a still image, and generation of the moving image. A configuration of the OCT apparatus in Embodiment 3 is similar to the OCT apparatus in Embodiment 1 and Embodiment 2. Therefore, the same reference symbols are used, and a description thereof is omitted. Now, the control unit 200 in Embodiment 3 is described mainly for differences from Embodiment 1 and Embodiment 2.

In Embodiment 3, three imaging modes including, in addition to a preview mode in which the preview image are displayed, and a still image mode in which the still image is displayed, a moving image mode in which the moving image is displayed is provided. In Embodiment 3, acquisition and processing conditions for the OCT signal are set so that the still image is higher in quality than the moving image, and the moving image is higher in quality than the preview image. Specifically, the acquisition condition setting unit 216 and the processing condition setting unit 225 change the amount of acquired data and the amount of processed data of the OCT signal depending on the imaging mode.

The moving image is formed of a plurality of frames, and hence takes time to generate. Therefore, in Embodiment 3, the acquisition condition setting unit 216 sets the OCT signal acquisition conditions for the moving image so that the amount of acquired data of the OCT signal for the moving image becomes smaller than the amount of acquired data of the OCT signal for the still image.

Further, it is required that the preview image be displayed in real time. Therefore, the processing condition setting unit 225 sets the OCT signal acquisition conditions for preview so that the amount of processed data of the OCT signal for preview becomes smaller than the amount of processed data of the OCT signal for the moving image. In Embodiment 3, the acquisition condition setting unit 216 sets, as the OCT acquisition conditions for the preview image, acquisition conditions similar to the OCT signal acquisition conditions for the moving image.

Through the above-mentioned processing, the control unit 200 in Embodiment 3 can speed up the generation of the moving image. Further, depending on the image to be captured, the image can be generated and displayed in appropriate processing time.

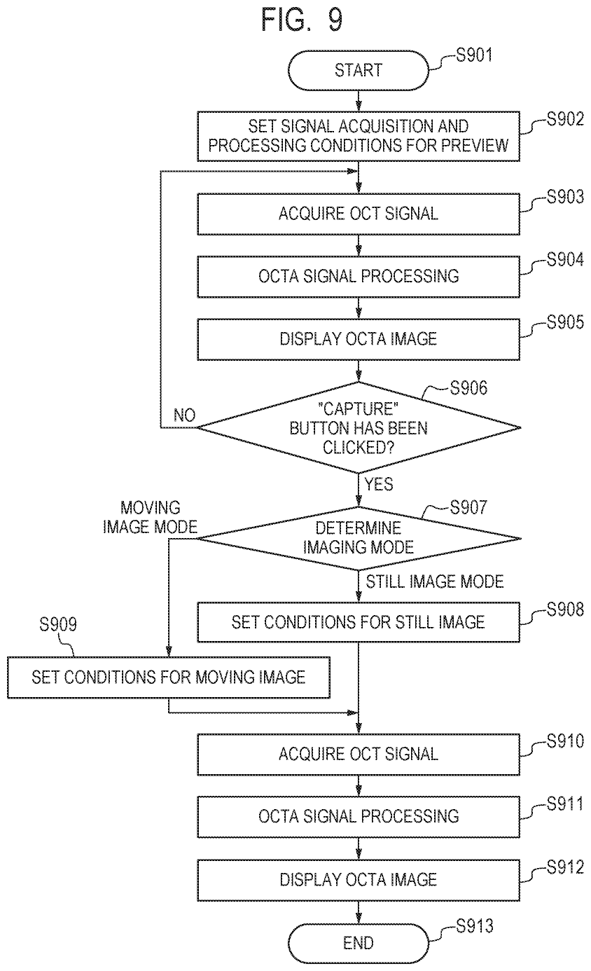

Now, referring to FIG. 9, the imaging sequence in Embodiment 3 is described. FIG. 9 is a flow chart of the imaging sequence in Embodiment 3. First, in Step S901 of FIG. 9, the control unit 200 detects that the examiner has clicked the “start moving image” button 301 on the screen 300 with the use of the operation input unit 170, and starts imaging.

In Step S902, the acquisition condition setting unit 216 and the processing condition setting unit 225 set the OCT signal acquisition conditions and the OCT signal processing conditions for the preview image (moving image). Here, the acquisition condition setting unit 216 sets the conditions such as the number “m” of A-scans so that the amount of acquired data of the OCT signal for the preview image becomes smaller than the amount of acquired data of the OCT signal for the still image. Further, the processing condition setting unit 225 sets the conditions such as the number “m” of A-scans so that the amount of processed data of the OCT signal for the preview image becomes smaller than the amount of processed data of the OCT signal for the still image and for the moving image. The setting of the values of the conditions is similar to Step S702 in Embodiment 1, and hence a description thereof is omitted.

In Step S903, the signal acquiring unit 210 acquires the OCT signal in accordance with the set acquisition conditions and the above-mentioned OCT signal acquiring sequence. In Step S904, the signal processing unit 220 performs the OCTA signal processing in accordance with the set processing conditions and the above-mentioned OCTA signal processing sequence to generate an OCTA image. The processing of Step S905 and Step S906 is similar to Steps S705 and S706 in Embodiment 1, and hence a description thereof is omitted. In Embodiment 3, a “capture” button used to issue an instruction to start capturing a still image or a moving image is provided instead of the “capture still image” button 302.

In Step S907, the control unit 200 determines which of the still image mode and the moving image mode is selected. The still image mode or the moving image mode can be selected with an imaging mode selection button (not shown) displayed on the screen 300, for example.

In Step S907, when the control unit 200 determines that the still image mode is selected, the processing proceeds to Step S908. In Step S908, the acquisition condition setting unit 216 and the processing condition setting unit 225 set the OCT signal acquisition conditions and the OCT signal processing conditions for the still image. The acquisition condition setting unit 216 and the processing condition setting unit 225 set the conditions such as the number “m” of A-scans so that the amount of acquired data and the amount of processed data of the OCT signal for the still image become larger than the amount of acquired data and the amount of processed data of the OCT signal for the preview image and for the moving image. Thereafter, the processing proceeds to Step S910.

In contrast, when the control unit 200 determines in Step S907 that the moving image mode is selected, the processing proceeds to Step S909. In Step S909, the acquisition condition setting unit 216 sets the OCT signal acquisition conditions for the moving image. Here, the acquisition condition setting unit 216 sets the conditions such as the number “m” of A-scans so that the amount of acquired data of the OCT signal for the moving image becomes smaller than the amount of acquired data of the OCT signal for the still image. Thereafter, the processing proceeds to Step S910.

In Step S910, the signal acquiring unit 210 acquires the OCT signal in accordance with the set acquisition conditions and the above-mentioned OCT signal acquiring sequence. In Step S911, the signal processing unit 220 performs the OCTA signal processing in accordance with the set processing conditions and the above-mentioned OCTA signal processing sequence to generate an OCTA image. In the case of the moving image mode, the signal processing unit 220 performs the OCTA signal processing in accordance with the OCTA signal processing sequence based on the acquired OCT signal. The subsequent processing is similar to Steps S710 and S711 in Embodiment 1, and hence a description thereof is omitted.

As described above, in Embodiment 3, the data amount of the tomographic data used in generating one OCTA image when the OCTA image is to be generated as the preview image is smaller than the data amount of the tomographic data used in generating one OCTA image when the OCTA image is to be generated as the moving image. Therefore, the time required to generate the preview image can be sped up as compared to the time required to generate the moving image. As a result, the OCTA images can be generated and displayed in appropriate processing time depending on the image to be captured.

In Embodiment 3, in the moving image mode, only the acquisition condition setting unit 216 sets the OCT signal acquisition conditions so that the amount of acquired data of the OCT signal for the moving image becomes smaller than the amount of acquired data of the OCT signal for the still image. However, in the moving image mode, it is only required that the OCT signal acquisition conditions and the OCT signal processing conditions be set so that the processing time becomes shorter than in the still image mode. Therefore, in the moving image mode, only the processing condition setting unit 225 may set the OCT signal acquisition conditions so that the amount of processed data of the OCT signal for the moving image becomes smaller than the amount of processed data of the OCT signal for the still image. Alternatively, the acquisition condition setting unit 216 and the processing condition setting unit 225 may set the OCT signal acquisition conditions and the OCT signal processing conditions in the similar manner.

Similarly, in the preview mode, the acquisition condition setting unit 216 may set the OCT signal acquisition conditions so that the amount of acquired data of the OCT signal for preview becomes smaller than the amount of acquired data of the OCT signal for the moving image.

### MODIFICATION EXAMPLE

In Embodiment 1 to Embodiment 3 as described above, in displaying the preview image, an OCTA image generated based on tomographic data acquired in an imaging frame immediately preceding the image display is displayed. Here, when the amount of acquired data and the amount of processed data are reduced by decimation for generation of the preview image, the quality of the generated and displayed OCTA image is reduced.

To address this problem, in displaying the preview image, an OCTA image can be generated and displayed based on tomographic data obtained by averaging tomographic data acquired in the imaging frame immediately preceding the image display and an imaging frame before the imaging frame, to thereby suppress the reduction in quality. Further, a similar effect can be obtained when an OCTA image is generated and displayed, which is obtained by averaging an OCTA image generated in the immediately preceding imaging frame and an OCTA image generated in an imaging frame before the immediately preceding imaging frame. Further, the number of frames of data to be averaged is not limited to two frames of the immediately preceding frame and the frame before the immediately preceding frame, and may be set to any number depending on a desired configuration. Through averaging the data and images so that the processed frames of the signal involved in generating the image fall within the acquired frames of the signal as described above, real-time display can be performed.

According to Embodiment 1 to Embodiment 3 and the modification example as described above, the generation of the moving image of the OCTA images can be sped up.

According to Embodiment 1 to Embodiment 3 and the modification example as described above, the preview image is displayed before the still image and the moving image are captured, but a preview imaging mode in which the preview image is captured and displayed may be provided separately from the still image capturing mode and the moving image capturing mode.

The processing in Embodiment 1 to Embodiment 3 and the modification example as described above is not limited to the configuration of being performed based on the luminance values of the tomographic images. The above-mentioned various kinds of processing may be applied to the tomographic data including the OCT signal acquired by the imaging apparatus unit 100, a signal obtained by Fourier-transforming the OCT signal, a signal obtained by performing suitable processing on the signal, and the tomographic images based on those signals. Also in those cases, similar effects as those obtained by the above-mentioned configuration can be obtained.

Further, in Embodiment 1 to Embodiment 3 as described above, the signal acquiring unit 210 acquires the OCT signal acquired by the imaging apparatus unit 100, the tomographic data generated by the OCT tomographic image generating unit 221, and other signals. However, the configuration in which the signal acquiring unit 210 acquires those signals is not limited thereto. For example, the signal acquiring unit 210 may acquire those signals from a server or an imaging apparatus connected to the control unit 200 via a LAN, a WAN, the Internet, or other networks.

Further, in Embodiment 1 to Embodiment 3 as described above, a configuration of a Michelson interferometer is used as an interferometer, but the configuration of the interferometer is not limited thereto. For example, the interferometer of the OCT apparatus 1 may have a configuration of a Mach-Zehnder interferometer. Further, a fiber optical system using the coupler is used as a splitting device, but a spatial optical system that uses a collimator and a beam splitter may also be used. Further, the configuration of the imaging apparatus unit 100 is not limited to the above-mentioned configuration, and a part of components included in the imaging apparatus unit 100 may be provided separately from the imaging apparatus unit 100.

Further, while the spectral-domain OCT (SD-OCT) apparatus, which uses the SLD as the light source 110, is described as the OCT apparatus 1 in Embodiment 1 to Embodiment 3 as described above, the configuration of the OCT apparatus according to the present invention is not limited thereto. For example, the present invention is also applicable to a swept-source OCT (SS-OCT) apparatus, which uses a wavelength-swept light source capable of sweeping a wavelength of emitted light, or other such freely-selected type of OCT apparatus.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An image processing apparatus comprising:
an acquiring unit configured to acquire a plurality of pieces of tomographic data indicating tomographic information at substantially the same position of a subject to be inspected;
a data generating unit configured to generate motion contrast data using the plurality of pieces of tomographic data;
a designating unit used to designate a range in a depth direction in generating a motion contrast image;
an image generating unit configured to generate the motion contrast image based on data in the range of the motion contrast data; and
a control unit configured to display, on a display unit, the motion contrast image, and an imaging region from which a still image is to be captured on the motion contrast image,
wherein the designating unit is also used to change the displayed imaging region, and
wherein a data amount of a plurality of pieces of tomographic data used in generating the motion contrast data when the motion contrast image is to be generated as a moving image is smaller than a data amount of a plurality of pieces of tomographic data used in generating the motion contrast data when the motion contrast image is to be generated as a still image.

2. The image processing apparatus according to claim 1, wherein a data amount of the plurality of pieces of tomographic data acquired by the acquiring unit to generate the motion contrast data in the case where the moving image is to be generated is smaller than a data amount of the plurality of pieces of tomographic data acquired to generate the motion contrast data when the still image is to be generated.

3. The image processing apparatus according to claim 2, wherein the acquiring unit is configured to set at least one value of a number of A-scans, a number of sets of B-scans, a number of repetitions of scans at substantially the same position, a sampling number of an interference signal, and a sampling range of the interference signal in acquiring the plurality of pieces of tomographic data to generate the motion contrast data when the moving image is to be generated, to be smaller than a value corresponding to the at least one value in acquiring the plurality of pieces of tomographic data to generate the motion contrast data when the still image is to be generated.

4. The image processing apparatus according to claim 1, wherein the image generating unit is configured to use, as the plurality of pieces of tomographic data to be processed to generate the motion contrast data when the moving image is to be generated, a part of the plurality of pieces of tomographic data acquired by the acquiring unit to generate the motion contrast data.

5. The image processing apparatus according to claim 4, wherein the image generating unit is configured to use, when the moving image is to be generated, of the plurality of pieces of tomographic data acquired by the acquiring unit to generate the motion contrast data, tomographic data based on a value obtained by reducing at least one value of a number of A-scans, a number of sets of B-scans, a number of repetitions of scans at substantially the same position, a sampling number of an interference signal, and a sampling range of the interference signal, to generate the motion contrast data.

6. The image processing apparatus according to claim 4, wherein the image generating unit is configured to use, when the moving image is to be generated, data obtained by decimating the plurality of pieces of tomographic data acquired by the acquiring unit, to generate the motion contrast data.

7. The image processing apparatus according to claim 4, wherein the image generating unit is configured to reduce, when the moving image is to be generated, an amount of the plurality of pieces of tomographic data used in generating the motion contrast data so that time required to process the plurality of pieces of tomographic data to generate the motion contrast data becomes shorter than time required to acquire the plurality of pieces of tomographic data to generate the motion contrast data.

8. The image processing apparatus according to claim 1, wherein the image generating unit is configured to generate, when the moving image is to be generated, a motion contrast image obtained by averaging a plurality of the motion contrast images as one motion contrast image of the moving image.

9. The image processing apparatus according to claim 8, wherein a data amount of a plurality of pieces of tomographic data used to generate the motion contrast data when the motion contrast image is to be generated as a preview image is smaller than the data amount of the plurality of pieces of tomographic data used to generate the motion contrast data when the motion contrast image is to be generated as the moving image.

10. An ophthalmic imaging apparatus comprising:
an imaging optical system configured to image substantially the same position of a subject to be inspected a plurality of times with use of measurement light to acquire a plurality of pieces of tomographic information at substantially the same position;
an acquiring unit configured to acquire a plurality of pieces of tomographic data indicating the plurality of pieces of tomographic information; and
a data generating unit configured to generate motion contrast data using the plurality of pieces of tomographic data,
wherein a data amount of a plurality of pieces of tomographic data used in generating the motion contrast data when the motion contrast image is to be generated as a moving image is smaller than a data amount of a plurality of pieces of tomographic data used in generating the motion contrast data when the motion contrast image is to be generated as a still image.

11. An image processing method comprising:
acquiring a plurality of pieces of tomographic data indicating tomographic information at substantially the same position of a subject to be inspected;
designating a range in a depth direction in generating a motion contrast image;
generating motion contrast data using the plurality of pieces of tomographic data;
generating a motion contrast image based on data in the range of the motion contrast data; and
displaying, on a display unit, the motion contrast image, and an imaging region from which a still image is to be captured on the motion contrast image,
wherein the displayed imaging region is changeable, and
wherein a data amount of a plurality of pieces of tomographic data used in generating the motion contrast data when the motion contrast image is to be generated as a moving image is smaller than a data amount of a plurality of pieces of tomographic data used in generating the motion contrast data when the motion contrast image is to be generated as a still image.

12. A non-transitory computer-readable medium having stored thereon a program for causing, when being executed by a computer, the computer to execute each step of the image processing method of claim 11.
